(19) United States
(12) Patent Application Publication
(10) Pub. No.: US 2017/0022558 A1
(43) Pub. Date: Jan. 26, 2017

(54) INTEGRATED SYSTEM FOR NUCLEIC ACID SEQUENCE AND ANALYSIS

(71) Applicant: Complete Genomics, Inc., Mountain View, CA (US)

(72) Inventors: William Banyai, San Francisco, CA (US); Scott Lockard, San Jose, CA (US); Daniel Chernikoff, Palo Alto, CA (US); David Rosenfeld, San Jose, CA (US); Arnold Oliphant, Morgan Hill, CA (US)

(21) Appl. No.: 15/171,872

(22) Filed: Jun. 2, 2016

Related U.S. Application Data
(60) Provisional application No. 62/171,879, filed on Jun. 5, 2015.

(51) Int. Cl. C12Q 1/68; G01N 35/00

(52) U.S. Cl. CPC C12Q 1/6874; G01N 35/0092; G01N 35/0099; G01N 35/00722; G01N 2035/0091

(57) ABSTRACT

An integrated end-to-end system for large-scale, high-quality nucleic acid sequencing having a nucleic acid extraction module, a library preparation module, a nucleic acid sequencing module, and a data analysis module reversibly integrated with one another and having components that are physically loosely-coupled within such system and reversibly integrated for sequence interrogation and analysis. This system is fully automated from sample to data output. A workflow management system is integrated across all system components and provides an intuitive user interface for managing system operations.

### CROSS-REFERENCES TO RELATED APPLICATIONS

This application claims priority benefit under 35 USC §119 of provisional application Ser. No. 62/171,879 filed Jun. 5, 2015. This application follows U.S. application Ser. No. 12/261,548, filed Oct. 30, 2008, which claims priority to provisional application 60/983,886, filed Oct. 30, 2007. The three aforesaid applications are hereby incorporated herein by reference in their entirety for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

The invention relates generally to the field of nucleic acid sequencing and analysis and more particularly to equipment and systems for high speed and high volume automation of the processes.

Numerous enterprises have approached the challenge of high throughput DNA sequencing with the development of DNA sequencing systems. Although such systems have decreased the cost and increased the efficiency of DNA sequencing, these systems are generally self-contained units with multiple interdependent components. Such single unit sequencing systems have numerous limitations, including limited scalability, a time lag in the introduction of innovations to specific components, and direct dependency of function of the entire system on each component of the system.

Recently, Illumina, Inc. introduced the HiSeq X Ten system, which consists of a set of ten HiSeq X ultra-high-throughput instruments. This system is intended to produce about 18,000 human genomes a year at 30× coverage. However, the HiSeq X Ten system suffers from various deficiencies. DNA sequencers produce reads, not interpretation-ready data. Additional time, expertise and expense are needed to convert reads to interpretable data. Also, system components for sample-to-data sequencing workflows require integration by the customer. A lack of automation of some steps in the end-to-end process results in manual processing due to lack of automation, increased cost and time, and decreased quality.

Moreover, in order to use such systems from sample to interpretation-ready data, multiple specialized competencies are required: biochemistry, bioinformatics, lab operations, IT subspecialties, etc.

The current invention addresses limitations of known prior art.

### BRIEF SUMMARY OF THE INVENTION

According to the invention, an integrated, end-to-end system for large-scale, high-quality nucleic acid sequencing is provided that combines high throughput, flexibility, scalability, and end-to-end automation by attention to workflow issues of inherently conflicting physical handling-, chemical reaction-, high-resolution imaging-, and analytical-processes.

This system is fully automated from sample to data output. It integrates DNA extraction, library preparation, sequencing, sequence assembly, and data analysis into a simple workflow. The system's Workflow Management System (WMS) is integrated across all system components and provides an intuitive user interface for managing system operations, allowing users to monitor full system status from a single screen.

Systems according to the invention comprise multiple, purpose-based, discrete components that are physically loosely-coupled within such system and reversibly integrated for sequence interrogation and analysis. By physically loosely-coupled it is meant that the components are spaced apart and/or physically isolated by vibration isolation mechanisms such as shock absorbers or dampers and springs while being physically juxtaposed in operation. This is facilitated by a control and sensing mechanism as part of a carrying mechanism as well as a position registration mechanism as part of the analysis and characterization mechanism so that the components and modules can be close and interactive without vibrational interference. By “reversibly integrated” it is meant that modules and components are physically connected to one another in such a manner that they can be readily removed and replaced. This may be facilitated by and may require standardized interconnection interfaces for the modules and components. The loosely coupled and reversible integrated nature of the system provides greater efficiency and versatility in the use of the various system components, allowing optimization of the system based on the time requirements and the capabilities of each component. This allows for improved flexibility, scalability, ease of maintaining, repairing or adding improvements to the system, and the creation of multiple system configurations with an enhanced user flexibility compared to fully integrated systems presently available in the art. Having the system elements loosely coupled and reversibly integrated provides numerous benefits, including facilitating any repairs that need to be made in a single component of the system while not disrupting the other components overall system. In addition, the coupling strategy of the individual system components facilitates the introduction of any improvement to a single component, thus promoting the use of new innovations and providing the latest state of the art innovations to the overall system.

According to one embodiment of the invention, an integrated, automated nucleic acid sequencing system is provide that comprises: (a) a nucleic acid extraction module, wherein a nucleic acid is extracted from a sample that comprises the nucleic acid; (b) a library preparation module, wherein a library of barcoded nucleic acid constructs is prepared from the extracted nucleic acid; (c) a nucleic acid sequencing module comprising a flow cell loader, at least one (one, two, three or more) flow cell, an imager, and at least one (one, two, three or more) liquid handler that performs sequencing reactions, wherein (i) the flow cell(s) comprises a substrate for attachment of the barcoded nucleic acid constructs in an array, (ii) the flow cell loader is configured to load the barcoded nucleic acid constructs into the flow cell, (iii) the liquid handler(s) is configured to perform nucleic acid sequencing reactions on the barcoded nucleic acid constructs in the array, and (iv) the imager is configured to produce images of the barcoded nucleic acid constructs in the array after sequencing; (d) a data analysis module, wherein the images are analyzed to produce reads, the reads are assembled to produce assembled sequence, and variants are identified in the assembled sequence; and (e) a workflow management system comprising a user interface for managing operation of the nucleic acid extraction module, the library preparation module, the nucleic acid sequencing module and the data analysis module.

In specific embodiments of the invention, higher throughput can be achieved by using multiple components in the performance of the respective activities needed for nucleic acid sequencing. For example, using multiple optical detection instruments and/or multiple sequencing reaction components can greatly increase the number of sequences determined and decrease the time required for doing so. In one embodiment, the nucleic acid sequencing module comprises a plurality (two or more) of liquid handlers, which optionally operate independently of each other and which can perform their functions according to different schedules, i.e., asynchronously. For example, one liquid handler may perform a first type of sequencing reaction (e.g., cPAL sequencing) and another liquid handler may perform a second type of sequencing reaction that differs from the second sequencing reaction (e.g., sequencing by synthesis). Alternatively, all liquid handlers may perform the same type of sequencing reaction. In another embodiment, the nucleic acid sequencing module comprises a plurality of flow cells.

In another embodiment, the liquid handler and the imager are loosely coupled, the system comprising a carrying device configured for transferring said at least one flow cell from the liquid handler to the imager.

In another embodiment, the nucleic acid sequencing module and the imager are configured to operate independently at different rates.

In another embodiment, the nucleic acid sequencing module comprises shock isolators that are constructed and arranged so as to sufficiently isolate the imager from vibrations so that the vibrations do not disrupt image capture by the imager.

In another embodiment, such a nucleic acid sequencing system is employed in a method comprising: extracting a nucleic acid from a sample comprising the nucleic acid using the nucleic acid extraction module; preparing a library of barcoded nucleic acid constructs from the extracted nucleic acid using the library preparation module; loading the library of nucleic acid constructs into said at least one flow cell comprising a substrate for attachment of the constructs in an array using the flow cell loader; performing nucleic acid sequencing reactions on the nucleic acid constructs in said at least one flow cell; producing images of the nucleic acid constructs in the array after sequencing using the imager; performing data analysis using the data analysis module, wherein a basecalling element operating a data processing component produces reads from analysis of the images, a sequence assembly element assembles the reads to produce an assembled sequence, and a variant identification element identifies variants in the assembled sequence; and managing the workflow from extracting the nucleic acid to data analysis using the workflow management system.

According to another embodiment of the invention, methods of nucleic acid sequencing are provided that comprise a fully automated workflow that comprises the steps of: (a) extracting a nucleic acid from a sample comprising the nucleic acid; (b) preparing a library of barcoded nucleic acid constructs from the extracted nucleic acid; (c) loading the library of nucleic acid constructs into at least one flow cell comprising a substrate for attachment of the constructs in an array; (d) performing nucleic acid sequencing reactions on the nucleic acid constructs in said at least one low cell; producing images of the nucleic acid constructs after sequencing; (e) performing data analysis comprising producing reads from analysis of the images, assembling the reads to produce an assembled sequence, and identifying variants in the assembled sequence; and (f) managing operation of the workflow using a workflow management system comprising a user interface.

In one embodiment, such a method comprises extracting nucleic acids from a plurality of samples; preparing separate libraries of nucleic acid constructs from each of said plurality of samples; pooling said separate libraries; and loading the pooled libraries into one or a plurality of flow cells.

In another embodiment, the method comprises amplifying the library of nucleic acid constructs to produce DNA nanoballs before loading into the flow cell(s). In another embodiment, nucleic acid constructs are amplified after loading into the flow cell(s). In another embodiment, nucleic acid constructs are amplified both before loading (e.g., to produce DNA nanoballs), and after loading (e.g., once an array of DNA nanoballs is formed post-loading, the DNA nanoballs are further amplified in situ in the flow cell(s)).

In one of the embodiments, a single reaction apparatus for sequencing and a single optical detection and analysis instrument are provided, with the reaction apparatus being physically loosely coupled and reversibly integrated with the optical instrument. In other embodiments, faster performance and higher throughput can be achieved by using multiple modules or individual module components in the performance of the respective activities needed for nucleic acid sequencing. This approach is useful in minimizing bottlenecks when different steps in the overall workflow, e.g., the step of performing nucleic acid sequencing reactions in the flow cells and the step of producing images of the nucleic acid constructs after sequencing, operate at different rates and/or according to different schedules, i.e., asynchronously. The workflow management system manages the overall workflow in order for the process to operate smoothly and efficiently and to reduce or eliminate bottlenecks.

For example, in one embodiment, multiple biochemistry components and a single optical detection instrument are provided for use with different sequencing reaction components, e.g., components directed to sequencing by synthesis and components directed to sequencing by probe ligation or cPAL sequencing. The sequencing reaction components of such systems can be kept in discrete units, with each unit reversibly interconnected physically to an optical imaging system. This allows a single system to utilize different sequencing technologies and benefit from the strengths of multiple different sequencing approaches in a single device configuration. The optical instrument can be disposed in a single system having an analysis component, or they may be deployed as two separate components of the overall system.

In one embodiment, the step of performing nucleic acid sequencing reactions on the nucleic acid constructs is performed using one or more (one or a plurality, i.e., two, three or more) liquid handler(s) that may operate independently of each other. In another embodiment, the step of performing nucleic acid sequencing reactions on the nucleic acid constructs is performed using a plurality of liquid handlers, and each of the flow cells is serially transferred to an imager for producing images of the nucleic acid constructs after sequencing.

In another embodiment, the step of producing images of the nucleic acid constructs after sequencing is performed using an imager, and the method further comprises transferring said at least one flow cell from the liquid handler to the imager using a carrying device.

In one specific embodiment, the system may comprise three compartmentalized components: (i) a fluidics system for storing and transferring detection and processing reagents, e.g., probes, wash solutions, and the like; (ii) a reaction platform for carrying out the biochemical sequencing reactions in a series of reaction chambers, or flow cell(s); and (iii) a discrete illumination and detection system for capture of optical images of the sequencing reactions and analysis of such images.

The reaction platform for the biochemical sequencing reactions preferably has multiple reaction units comprising individual flow cells and a mechanism for transfer of each flow cell from the reaction apparatus to the illumination and detection system following completion of the biochemical sequencing reaction.

Flow cells for sequencing reaction and analysis are known. Examples of such flow cells include those comprising any substrate used for the performance of a sequencing reaction, such as those described in more detail herein, as well as those described in U.S. Pat. Nos. 5,958,760, 6,403,376, 6,960,437, 7,025,935, 7,118,910, 7,220,549, 7,244,559, 7,264,929, WO 01/35088, and Published U.S. Patent App. 2007/0128610. In a preferred aspect of multiple embodiments, the flow cells comprise an array of nucleic acids of unknown sequence attached to a solid surface, e.g., glass or a flexible material such as a film or membrane. In another embodiment, each flow cell comprises an array of nucleic acids of unknown sequence attached to beads which are optionally attached to a solid or semi-solid surface.

In a certain aspect of the embodiments of the invention, the sequencing reaction component of the system provides a plurality of flow cells for use in processing a sample. In a preferred aspect, each flow cell comprises a substantially sealed chamber with a fluid inlet and a fluid outlet for the introduction and removal respectively of fluids used in the sequencing reaction.

In a specific embodiment, two or more sequencing reaction platforms can be interconnected to a single optical imaging system, which can record and analyze the separate sequencing information from each reaction unit. In a specific aspect, each of the reaction units and flow cells on the multiple reaction platforms are designed to carry out the same high throughput nucleic acid sequencing biochemistry on a plurality of flow cells. In another aspect, the different reaction platforms and flow cells are designed to accommodate different biochemical approaches to high throughput nucleic acid sequencing, with each reaction platform optimized to carry out a specific flow cell sequencing reaction. The ability to have optimized reaction platforms and flow cell biochemical reaction units, each designed to accommodate the specific biochemistry of a sequencing approach, reversibly interconnected with a single illumination and analysis system provides optimum use of space and run time and is more cost effective than having separate complete systems for each potential biochemical sequencing application.

In a particular aspect of certain embodiments, part of the internal surface of each of the flow cells is defined by the sample-bearing surface of the support, which arrangement has the advantage of minimizing the number of components involved in the flow cell assembly.

In a specific embodiment, the flow cells of a specific sequencing reaction unit each comprise an array of target nucleic acids of unknown sequence by sandwiching the glass and a gasket between two solid planar surfaces. One plane has an opening of sufficient size to permit imaging, and an indexing pocket for the cover slip. The other plane has an indexing pocket for the gasket, fluid ports, and an optional temperature control system.

In one specific aspect of the invention, a flow cell designed for specific use with a sequencing reaction unit comprises a 1″square, 170 micrometer thick cover slip. In a preferred embodiment, this flow cell has one surface that has been derivatized to bind macromolecular biologic structures of unknown sequence for high throughput, genome-scale sequencing.

In certain specific aspects of the invention, the flow cells may comprise a fluid port connected to a device (e.g., a syringe pump) with the ability to effect exit or entry of fluid from the flow cell.

In another specific aspect of the invention, the flow cell comprises a port connected to a mixing chamber, which is optionally equipped with a liquid level sensor. Solutions needed for the sequencing reaction are dispensed into the chamber, mixed if needed, then drawn into the flow cell. In a preferred aspect, the chamber is conical in nature and acts as a funnel. In certain aspects of the embodiments of the invention, each flow cell comprises a temperature control subsystem with ability to maintain temperature in the range from about 5-95° C., or more specifically 10-85° C., and can change temperature with a rate of about 0.5-2° C. per second.

In a further aspect of certain embodiments of the invention, the system further provides an automated apparatus for processing a sample, especially a biological sample, supported on a support, the apparatus comprising: support holding means for holding one or more supports, the sample on the or each support being present within a respective substantially sealed chamber; fluid delivery means for delivering processing fluid to the or each chamber; waste fluid collecting means for removing fluid from the or each chamber; and computer control means for monitoring the sequencing reaction. Preferably the apparatus is used in conjunction with one or more of the flow cells defined above.

The invention will be better understood to those persons skilled in the art upon reading the details of the methods as more fully described below.

### BRIEF DESCRIPTION OF THE DRAWINGS

FIG. 1 is a graphic representation of the modules of a fully integrated sequencing system for high throughput genomics.

FIG. 2 shows an embodiment of a library preparation module, including a liquid handler, sample and reagent storage, a spectrophotometer, plate sealers and peelers, thermal cyclers and consumable storage.

FIG. 3 shows an embodiment of a sequencing module, including a robot, an imager, electronics, three liquid handlers, flow cell storage, and a flow cell loader (not shown).

FIG. 4 shows an imager from the sequencing module, which is isolated from vibrations by means of shock isolators.

FIG. 5 shows a robot from the sequencing module, which includes a gripper at the end of the robotic arm for grasping flow cells.

FIG. 6 is a graphic drawing illustrating the basic format of a sequencing reaction platform of the invention.

FIG. 7 is a graphic drawing illustrating a first embodiment of a system comprising a sequencing reaction platform and an optical imaging device.

FIG. 8 is a graphic drawing illustrating a second embodiment of a system comprising a sequencing reaction platform and an optical imaging device.

FIG. 9 is a graphic drawing illustrating a third embodiment of a system comprising a sequencing reaction platform comprising a telescopic arm and an optical imaging device.

FIG. 10 is a graphic drawing illustrating a system comprising a parallel configuration of sequencing reaction platforms.

FIG. 11 is a graphic drawing illustrating a system in which the sequencing reaction component comprises one or more flow cells (or reaction chambers).

### DETAILED DESCRIPTION OF THE INVENTION

### Definitions

In order to have a sufficient background in the present technology, it is helpful to understand the following terms of art.

“Amplicon” means the product of a polynucleotide amplification reaction, namely, a population of polynucleotides that are replicated from one or more starting sequences. Amplicons may be produced by a variety of amplification reactions, including but not limited to polymerase chain reactions (PCRs), linear polymerase reactions, nucleic acid sequence-based amplification, rolling circle amplification and like reactions (see, e.g., U.S. Pat. Nos. 4,683,195; 4,965,188; 4,683,202; 4,800159; 5,210,015; 6,174,670; 5,399,491; 6,287,824 and 5,854,033; and US Pub. No. 2006/0024711).

“Array” or “microarray” refers to a solid support having a surface, preferably but not exclusively a planar or substantially planar surface, which carries a collection of sites comprising nucleic acids such that each site of the collection is spatially defined and not overlapping with other sites of the array; that is, the sites are spatially discrete. The array or microarray can also comprise a non-planar interrogatable structure with a surface such as a bead or a well. The oligonucleotides or polynucleotides of the array may be covalently bound to the solid support, or it may be non-covalently bound. Conventional microarray technology is reviewed in, e.g., Schena, Ed. (2000), Microarrays: A Practical Approach (IRL Press, Oxford). As used herein, “random array” or “random microarray” refers to a microarray where the identity of the oligonucleotides or polynucleotides is not discernable, at least initially, from their location but may be determined by a particular biochemistry detection technique on the array. See, e.g., U.S. Pat. Nos. 6,396,995; 6,544,732; 6,401,267; and 7,070,927; WO publications WO 2006/073504 and 2005/082098; and US Pub Nos. 2007/0207482 and 2007/0087362.

“Hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1M, more usually less than about 500 mM and less than about 200 mM. A “hybridization buffer” is a buffered salt solution such as 5×SSPE, or the like. Hybridization temperatures can be as low as 5° C., but are typically greater than 22° C., more typically greater than about 30° C., and preferably in excess of about 37° C. Hybridizations are usually performed under stringent conditions, i.e., conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tm for the specific sequence at a defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning: A laboratory Manual” 2nd Ed. Cold Spring Harbor Press (1989) and Anderson “Nucleic Acid Hybridization” 1st Ed., BIOS Scientific Publishers Limited (1999).

“Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

“Ligation” means to form a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon of a terminal nucleotide of one oligonucleotide with a 3′ carbon of another oligonucleotide. A variety of template-driven ligation reactions are described in the following references: Whitely et al, U.S. Pat. No. 4,883,750; Letsinger et al, U.S. Pat. No. 5,476,930; Fung et al, U.S. Pat. No. 5,593,826; Kool, U.S. Pat. No. 5,426,180; Landegren et al, U.S. Pat. No. 5,871,921; Xu and Kool, Nucleic Acids Research, 27: 875-881 (1999); Higgins et al, Methods in Enzymology, 68: 50-71 (1979); Engler et al, The Enzymes, 15: 3-29 (1982); and Namsaraev, U.S. patent publication 2004/0110213. Enzymatic ligation usually takes place in a ligase buffer, which is a buffered salt solution containing any required divalent cations, cofactors, and the like, for the particular ligase employed.

“Mismatch” means a base pair between any two of the bases A, T (or U for RNA), G, and C other than the Watson-Crick base pairs G-C and A-T. The eight possible mismatches are A-A, T-T, G-G, C-C, T-G, C-A, T-C, and A-G.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following process: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each reaction condition in a thermal cycler instrument. Particular temperatures, durations and rates of change between reactions depend on many factors well-known to those of ordinary skill in the art, e.g. exemplified by the references: McPherson et al., editors, PCR: A Practical Approach and PCR2: A Practical Approach (IRL Press, Oxford, 1991 and 1995, respectively). For example, in a conventional PCR using Taq DNA polymerase, a double-stranded target nucleic acid may be denatured at a temperature >90° C., primers annealed at a temperature in the range 50-75° C., and primers extended at a temperature in the range 72-78° C. As above, the term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g., 200 nL, to a few hundred μL, e.g., 200 μL.

“Nucleic acid” and “oligonucleotide” are used herein to mean a polymer of nucleotide monomers. As used herein, the terms may also refer to-double stranded forms. Monomers making up nucleic acids and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like, to form duplex or triplex forms. Such monomers and their internucleosidic linkages may be naturally occurring or may be analogs thereof, e.g., naturally occurring or non-naturally occurring analogs. Non-naturally occurring analogs may include peptide nucleic acids, locked nucleic acids, phosphorothioate internucleosidic linkages, bases containing linking groups permitting the attachment of labels, such as fluorophores, or haptens, and the like. Whenever the use of an oligonucleotide or nucleic acid requires enzymatic processing, such as extension by a polymerase, ligation by a ligase, or the like, one of ordinary skill would understand that oligonucleotides or nucleic acids in those instances would not contain certain analogs of internucleosidic linkages, sugar moieties, or bases at any or some positions, when such analogs are incompatible with enzymatic reactions. Nucleic acids typically range in size from a few monomeric units, e.g., 5-40, when they are usually referred to as “oligonucleotides,” to several hundred thousand or more monomeric units. Whenever a nucleic acid or oligonucleotide is represented by a sequence of letters (upper or lower case), such as “ATGCCTG,” it will be understood that the nucleotides are in 5′□3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually nucleic acids comprise the natural nucleosides (e.g., deoxyadenosine, deoxycytidine, deoxyguanosine, deoxythymidine for DNA or their ribose counterparts for RNA) linked by phosphodiester linkages; however, they may also comprise non-natural nucleotide analogs, e.g., modified bases, sugars, or internucleosidic linkages. To those skilled in the art, where an enzyme has specific oligonucleotide or nucleic acid substrate requirements for activity, e.g., single-stranded DNA, RNA/DNA duplex, or the like, then selection of appropriate composition for the oligonucleotide or nucleic acid substrates is well within the knowledge of one of ordinary skill, especially with guidance from treatises, such as Sambrook et al., Molecular Cloning, Second Edition (Cold Spring Harbor Laboratory, New York, 1989), and like references.

“Primer” means an oligonucleotide, either natural or synthetic, which is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually primers are extended by a DNA polymerase. Primers usually have a length in the range of from 9 to 40 nucleotides, or in some embodiments, from 14 to 36 nucleotides.

“Probe” as used herein refers to an oligonucleotide, either natural or synthetic, which is used to interrogate complementary sequences within a nucleic acid of unknown sequence. The hybridization of a specific probe to a target polynucleotide is indicative of the specific sequence complementary to the probe within the target polynucleotide sequence.

“Readout” means a parameter, or parameters, that are measured and/or detected and that can be expressed as a number, a value or other indicia for evaluation. In some contexts, readout may refer to an actual numerical representation of such collected or recorded data. For example, a readout of fluorescent intensity signals from a microarray is the position and fluorescence intensity of a signal being generated at each hybridization site of the microarray; thus, such a readout may be registered or stored in various ways, for example, as an image of the microarray, as a table of numbers, or the like.

“Solid support” and “support” are used interchangeably and refer to a material or group of materials having a rigid or semi-rigid surface or surfaces. Microarrays usually comprise at least one planar solid phase support, such as a glass microscope slide.

As used herein, the term “Tm” is used in reference to the “melting temperature.” The melting temperature is the temperature at which a population of double-stranded nucleic acid molecules becomes half dissociated into single strands. Several equations for calculating the Tm of nucleic acids are well known in the art. As indicated by standard references, a simple estimate of the Tm value may be calculated by the equation, Tm=81.5+0.41 (% G+C), when a nucleic acid is in aqueous solution at 1M NaCl (see e.g., Anderson and Young, Quantitative Filter Hybridization, in Nucleic Acid Hybridization (1985). Other references (e.g., Allawi, H. T. & SantaLucia, J., Jr., Biochemistry 36, 10581-94 (1997)) include alternative methods of computation which take structural and environmental, as well as sequence characteristics into account for the calculation of Tm.

By way of explanation, unless defined otherwise, all technical and scientific terms used herein have the same meaning as commonly understood by one of ordinary skill in the art to which this invention belongs.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Generally, and except where indicated, the molecular biology and sequencing analysis referred to with respect to the invention are, in their basic aspects, conventional methods within the skill of the art of those employed in the relevant field. Such techniques are explained fully in the literature, see, e.g., Maniatis, Fritsch & Sambrook, Molecular Cloning: A Laboratory Manual (1982); and Sambrook, Russell and Sambrook, Molecular Cloning: A Laboratory Manual (2001). Terms and symbols of nucleic acid chemistry, biochemistry, genetics, and molecular biology used herein follow those of standard treatises and texts in the field, e.g., Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

### Nucleic Acid Sequencing System

The inventors have developed an integrated, end-to-end genomics solution for large-scale, high-quality nucleic acid sequencing, including without limitation human genome and exome sequencing, that combines high throughput, flexibility, scalability, and end-to-end automation. A single system can support up to 10 sequencers at 10,000 whole human genomes sequenced per sequencer per year, or 100,000 genomes per year total. Thus, the system's scalability and throughput surpasses that of any other sequencing solution that is currently available.

The system herein described is fully automated from sample input to data output. The system fully integrates DNA extraction, library preparation, sequencing, sequence assembly, and data analysis into a simple workflow. Automation of the entire workflow provides multiple benefits, including reduced operational and staffing costs, lower error rates, higher data quality, and shorter turnaround time. As the system is already pre-configured and integrated, it eliminates the time and expense required to identify, purchase, integrate, and validate the systems components needed from multiple vendors. Full system integration provides numerous benefits. Time and money savings are provided at system start-up, since there is no need to purchase, integrate and validate separate components. The system can be installed and deployed more rapidly. Moreover, the system operates more efficiently, since it functions as a single application with dashboards for easy monitoring of system operations.

The system's Workflow Management System (WMS) is integrated across all system components and provides an intuitive user interface (UI) for managing system operations, allowing users to monitor full system status from a single screen. The operator can use the WMS to track samples, plan and execute workflows, manage exceptions, monitor the system as a whole, and generate reports. The API-centric design of the WMS also supports integration with customer systems and third-party applications. System monitoring is facilitated by a monitor dashboard, through which the operator is informed of system events, can audit sample processing, and can obtain reports on performance. The web-based design of the WMS allows remote planning and monitoring and provides web and tablet support. For example, in monitoring the sequencing module through the WMS, the operator can view the input queue, the in-process queue, obtain information about flow cell scans, etc.

According to one embodiment, the system performs whole-genome sequencing employing combinatorial probe-anchor ligation (cPAL) using mate pair libraries to deliver high confidence data on small variants, copy number variants and structural variants on about 97% of the human genome at 50× mean coverage. cPAL sequencing is described, for example, in U.S. Pat. Nos. 8,415,099; 8,518,640; and 8,551,702, and in Drmanac et al., Science 327:78-81, 2010.

Data is delivered in standardized file formats (FASTQ, BAM, VCF), to enable maximum compatibility with existing workflows, datasets, and interpretation platforms.

### The System and its Workflow

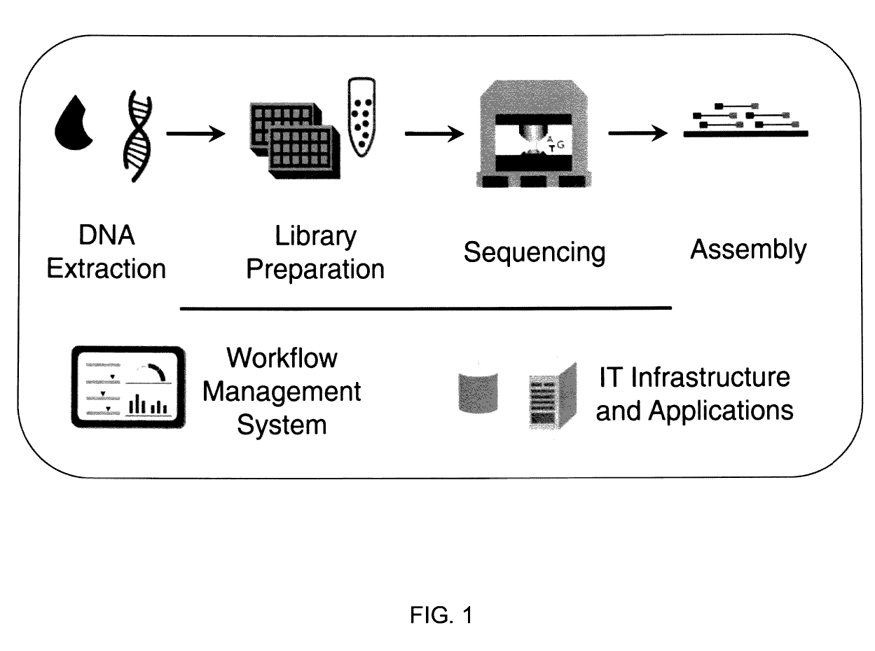

As shown in FIG. 1, the system includes all of the components necessary to perform DNA extraction, library preparation, sequencing, and data analysis, which are all coordinated using the Workflow Management System (WMS). There are four basic modules (or subsystems): a DNA extraction module, a library preparation module, a sequencing module and a data analysis (or assembly) module. The workflow management system (WMS) manages the workflow of a sample from sample input through DNA extraction, library preparation, and sequencing and the resulting data analysis. IT infrastructure and applications are also provided, including hardware for computation, data storage and network connectivity, and software for applications, operating systems, and database management infrastructure. In the DNA extraction module, genomic DNA (or other nucleic acids, e.g., mRNA) is extracted from a sample (e.g., blood or saliva). In the library preparation module, the nucleic acids are transformed into libraries of nucleic acid constructs that are ready for sequencing. In the sequencing module, the libraries are loaded into flow cells and sequenced to produce reads. In the data analysis module, the reads are assembled, variants are identified, and data output is provided in standardized formats.

The WMS provides workflow planning and execution, system monitoring, notification, and reporting functionalities that simplify and automate system-wide operations. It tracks samples, plans and executes workflows, manages system exceptions, monitors the system and generates reports. It uses a dashboard user interface (UI) that permits the operator to oversee the progress of samples and data, provides notifications, and instructs users on where samples are in the workflow.

The system also includes IT infrastructure and applications, including hardware for computation, data storage and network connectivity, and software for applications, operating systems, and database management infrastructure.

Any technology known in the art can be used for the modules, the WMS and the IT infrastructure and applications.

### DNA Extraction Module

The system's sample preparation module provides automated DNA purification from a variety of starting materials, including without limitation blood, saliva, fresh and preserved tissue (e.g., formalin-fixed, paraffin-embedded [FFPE] tissue), saliva, etc. Such samples may originate from humans or other organisms (mammals, birds, reptiles, fish, amphibians, higher plants, yeast, bacteria, etc.). The system processes numerous (e.g., 24-96) samples at a time using all-in-one reagent kits that minimize operator interaction with the system. An example of an automated DNA extraction module that can be used with the DNA extraction module is the QIAsymphony SP system, which includes all-in-one reagent kits (Qiagen). Other such systems are known in the art.

### Library Preparation Module

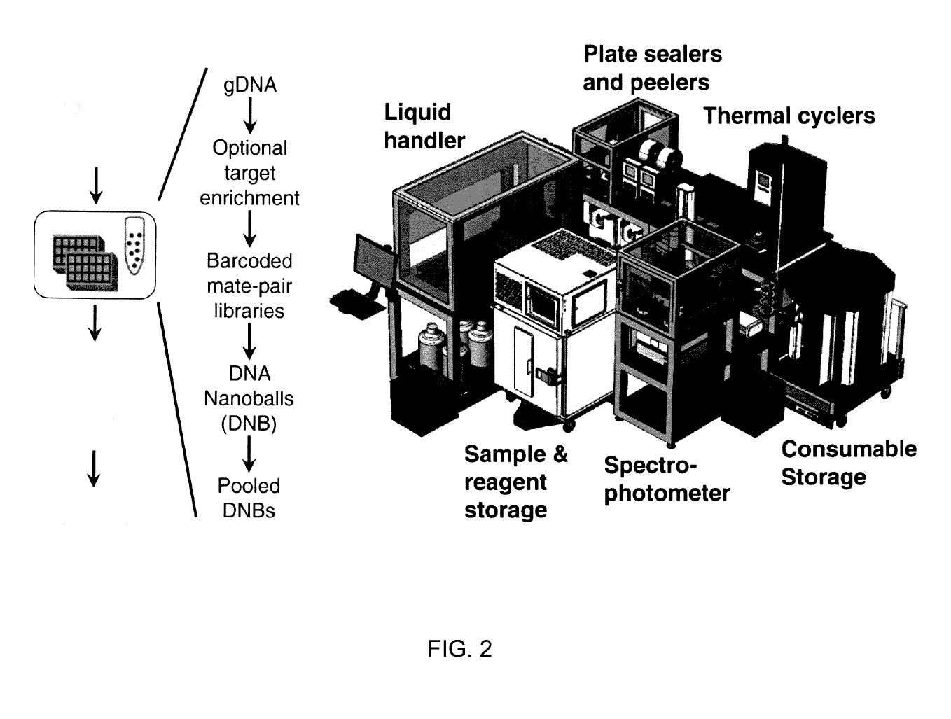

The system provides a fully-automated library preparation module designed to transform extracted genomic DNA (or other nucleic acids) into pooled mate-pair libraries containing barcoded sequencing substrates known as DNA nanoballs (DNBs), optionally including target enrichment. FIG. 2 shows an embodiment of a library preparation module of the systems of the invention, including a liquid handler, sample and reagent storage, a spectrophotometer, plate sealers and peelers, thermal cyclers and consumable storage. A mixture of inline quality control and planning functions ensures that only productive libraries are sequenced and maximizes flow-cell usage, driving flow cell usage to close to 100% and minimizing operating expenses. The system automation significantly reduces operator interaction to loading the system with samples, reagent kits, and plastics based on pre-defined workflows, thereby saving time and resources. The library preparation module includes such subcomponents as thermal cyclers, centrifuges, incubators, and ambient temperature and cooled storage for consumables. Samples, kits and consumables are stored onboard. The flexible design of the module supports future application. According to one embodiment, the module accepts 96-well microtiter plates and can re-array samples based on priority.

### DNA Sequencing Module

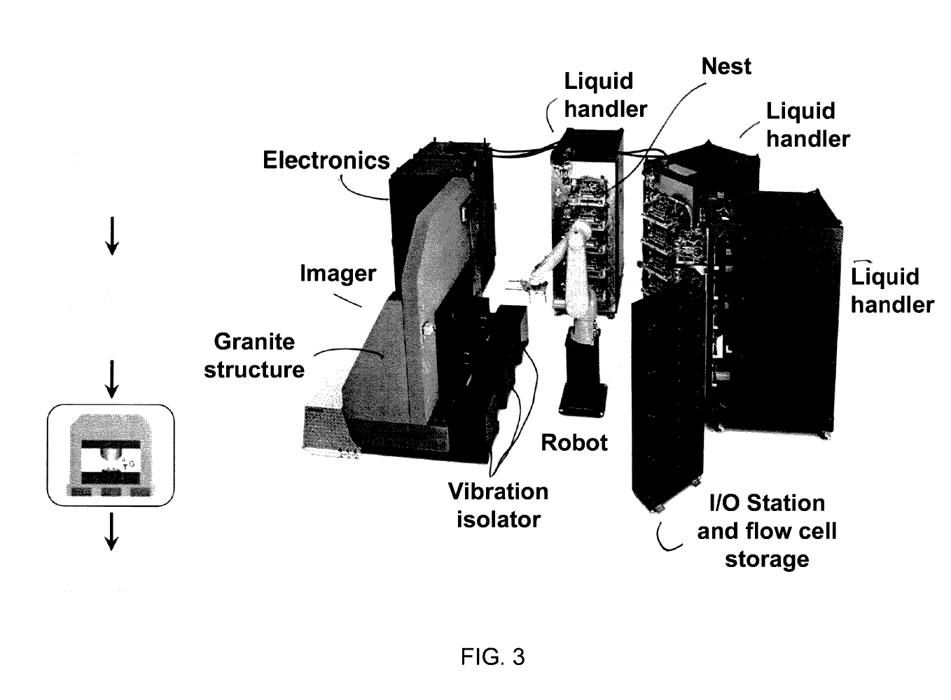

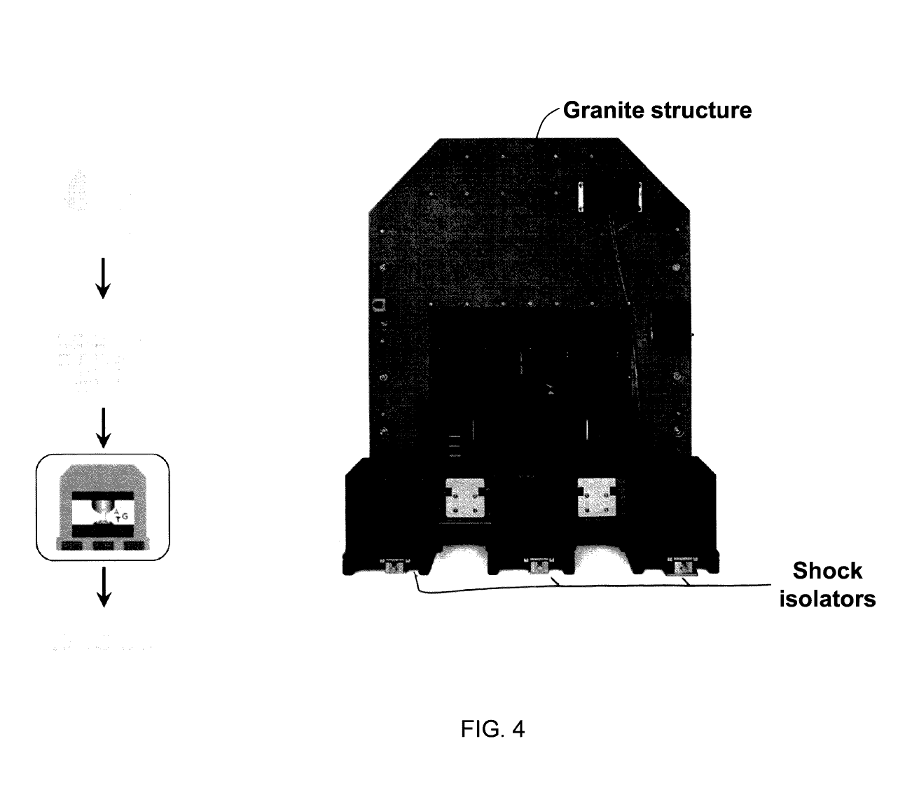

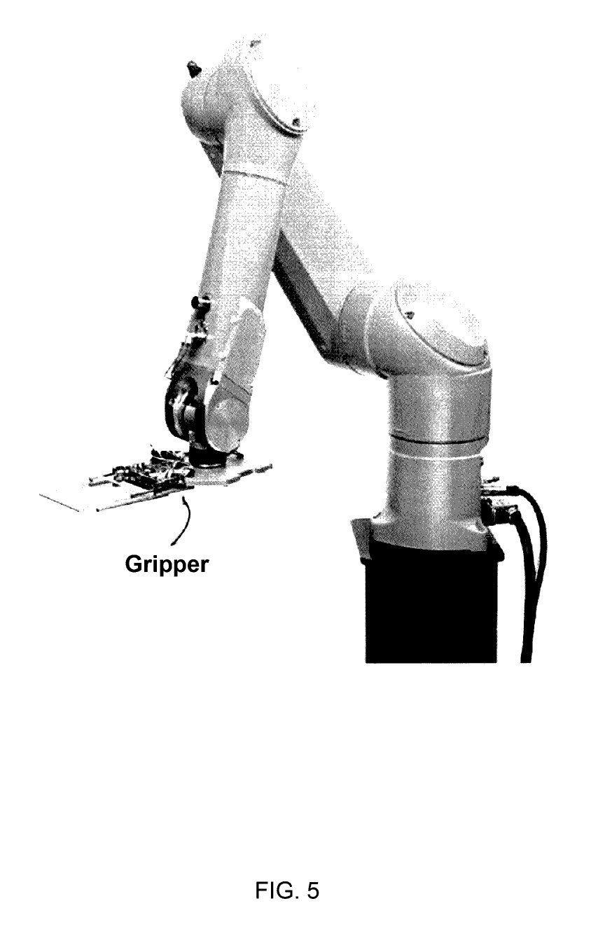

FIG. 3 shows an embodiment of a sequencing module, including a robot, an imager, electronics, three liquid handlers, flow cell storage, and a flow cell loader (not shown). According to one embodiment, each liquid handler processes up to four flow cells. FIG. 4 shows an imager, which includes shock isolators that isolate the imager from vibrations caused by moving parts in the other modules of the system or from the environment by means of shock isolators. FIG. 5 shows an industrial-scale robot from the sequencing module, which includes a gripper at the end of the robotic arm.

The barcoded DNBs are pooled and loaded into a flow cell provided with microfluidics that encloses a patterned silicon array that has a 48 billion spots, each spot providing an attachment site for a single DNB. Library construction for production of DNBs and random DNB arrays are described, for example, in U.S. Pat. Nos. 7,897,344; 7,901,890; 7,910,302; 7,910,354; 7,960,104; 8,133,719; 8,445,194; 8,445,196; 8,445,197; 8,440,397; 8,609,335; 8,722,326; and in Drmanac et al., Science 327:78-81, 2010. Flow cells are described, for example, in U.S. Patent Publication 2013/0281305 A1. Substrates for DNB arrays and their manufacture are discussed, for example, in U.S. Pat. Nos. 7,988,918; 8,287,812; and 8,765,359.

The system includes a flow cell loader and an ultra-high scale sequencing instrument consisting of a high-speed imager, an industrial-scale robot, and liquid handlers that perform the sequencing chemistry using cPAL technology.

Any imager that is known in the art may be used. Imagers and image registration technology for use in connection with the present invention are described, for example, for example, in U.S. Pat. Nos. 8,175,452; 8,428,454; 8,660,421; 8,774,494; U.S. Patent Publication No. 2012/0224050; 2014/0152793; and 2014/0152888. According to one embodiment, the high performance lens resolves features on the array at the wavelength of visible light and permits simultaneous four-color detection at high speed and resolution.

According to one embodiment the flow cells are provided with input and output ports for fluid flow and enclose patterned silicon arrays that have 48 billion spots for DNB attachment, with one DNB attaching at each spot. The design of the flow cell minimizes reagent use and speeds reagent exchange.

The liquid handlers operate independently of each other, providing the flexibility to stagger sequencing runs and to sequence different applications simultaneously. In addition to cPAL sequencing, for example, sequencing may be performed using a variety of sequencing methods known in the art, including, but not limited to,
hybridization-based methods, such as disclosed in Drmanac, U.S. Pat. Nos. 6,864,052; 6,309,824; and 6,401,267; and Drmanac et al, U.S. patent publication 2005/0191656.
sequencing-by-synthesis methods, such as disclosed in: Nyren et al, U.S. Pat. No. 6,210,891; Ronaghi, U.S. Pat. No. 6,828,100; Ronaghi et al., Science, 281: 363-365, 1998; Balasubramanian, U.S. Pat. No. 6,833,246; Quake, U.S. Pat. No. 6,911,345; Li et al, Proc. Natl. Acad. Sci., 100:414-419, 2003; and Smith et al, PCT publication WO 2006/074351.
ligation-based methods, e.g. Shendure et al., Science, 309:1728-1739, 2005; and Macevicz, U.S. Pat. No. 6,306,597.

Subcomponents of the sequencing module include a high-speed imager, an industrial-scale robotic arm, liquid handlers, an electronic rack, and flow cell storage. Each liquid handler normally processes up to four flow cells. Sequencing runs can be staggered on different liquid handlers, and multiple applications can be sequenced simultaneously (e.g., WGS or WES). The sequencing module is also designed to support multiple sequencing chemistries (cPAL, sequencing by synthesis, etc.) as noted above.

### Data Analysis Module

The data analysis module includes an element, typically software, for performing basecalling, mapping (if a reference-based assembly method is used), sequence assembly, and variant identification (including SNPs, insertions, deletions, block substitutions, rearrangements, copy number variations, etc. Any such software known in the art may be used. In one embodiment of the invention, assembly is performed by an assembler that uses a combination of Bayesian analysis and graph-based methods to perform local de novo assembly in regions of the genome most likely to be variant, and provides high confidence data on small variants, including SNPs, insertions, deletions, and block substitutions (U.S. Patent Publication No. 2011/0004413; Carnevali et al., J. Computational Biol. 19, 279-292, 2012). The data analysis module also identifies copy number variants and structural variations. The system also includes the applications, software, hardware, and database management infrastructure needed to support large-scale data management. Various basecallers are described in the art. Software for mapping short reads to a reference genome is described, for example, in U.S. Pat. Nos. 8,615,365; 8,731,843; and 8,738,296. Software for calling variations in a nucleic acid sequence with reference to a reference genome is described, for example, in U.S. Patent Publications No. 2011/0004413 and 2013/0110407. Software for detecting sequence rearrangements is described in U.S. Patent Publication No. 2012/0197533. Software for detecting copy number variants in a nucleic acid sequence is described, for example, in U.S. Patent Publications No. 2014/0229117. Variant calls are exported to industry standard file formats (e.g., VCF).

### IT Infrastructure and Applications

The system also includes IT infrastructure and applications, including hardware for computation (e.g., computers), data storage and network connectivity, and software for applications, operating systems, and database management infrastructure.

### System Workflow

Sequencing begins when the operator places DNB-loaded flow cells in the machine's Input/output (I/O) station. The dual-gripper robot, which has six degrees of freedom, transports the flow cell from the I/O station (specifically the input portion), which includes flow cell storage, to a liquid handler (or liquid handling system [LHS] rack), where the DNBs are tagged through a sequence of chemical cycles. Thereafter, the robot transports the flow cell from the liquid handler to the high-speed imager for subsequent florescence excitation and image collection. After imaging, the robot returns the flow cell to the liquid handler for an additional cycle of chemistry. After all sequencing cycles are completed, the robot transports the flow cell to the I/O station (specifically, the output portion), where it is ultimately retrieved by the operator.

The liquid handler houses four nests, a bulk reagent dispensing module (BRD; inside the liquid handler, not shown) and two low-volume reagent-dispensing modules (LVD; inside the liquid handler, not shown). The BRD consists of reagent-filled bottles, whose contents are fed to the nest via high-accuracy pumps and selector valves. Each LVD houses a three-axis robot that allows for dispensing anchors and probes via a needle/sample-loop aspiration mechanism to the nests. The nest is an electromechanically and pneumatically actuated assembly that serves as the interface between the flow cell and the liquid dispensing modules. The nest accepts the flow cell, and it allows for delivery of bulk and low-volume liquids through a dynamic sealing mechanism (or make/break seal). After sequencing reactions are performed, the robot transports the flow cell to the imager.

The imager consists of a vibration-isolated monolithic granite structure that supports two main components: a state-of-the-art custom-designed opto-mechanical assembly (backplane; not shown), a multi-axis air-bearing stage mechanism, and vibration isolators (or shock isolators). Excitation of the tagged DNBs and collection of the resulting fluorescence is achieved by conducting a highly precise serpentine motion of the flow cell while shining a laser beam on the sample. Simultaneously, the backplane's optical train is designed to image the fluorescent response of each DNB through an optical path consisting of a multi-element objective lens, which feeds light into four distinct camera channels.

### Exemplary Sequencing Module

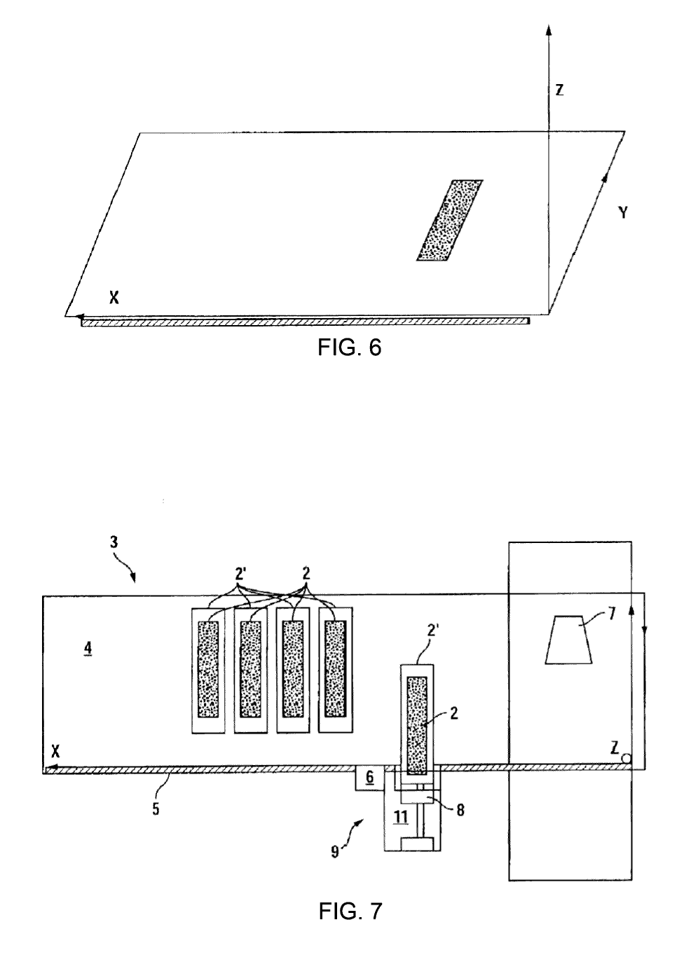

FIG. 6 shows a schematic side view of an exemplary sequencing reaction platform with a reaction workspace and having a lengthwise dimension X, a width dimension Y, and a height dimension Z.

FIG. 7 shows a schematic top view of a first sequencing reaction platform of one preferred aspect of the sequencing system embodiments of the invention. Platforms of this nature are also disclosed in U.S. Pat. No. 7,264,432. This reaction platform 3 comprises flow cells 2 placed on discrete solid supports 2′ and positioned on at least one essentially horizontal table 4 having lengthwise dimension X and a width dimension Y. This platform 3 includes at least one rail 5 extending parallel to the X direction and at least one displacement unit 6 having a carrying device 9, which is movable together with this displacement unit along the rail 5, for transferring objects in the X direction. The carrying device 9 is implemented here as a carrying plate 11, which is movable along the rail 5 together with the displacement unit 6, and a motorized gripping mechanism 8 for grasping and moving each discrete support 2 toward a coupled optical characterization tool 7. Using the gripping mechanism 8, the support 2′ and the flow cell 2 are pulled onto the carrying plate 11 and transferred toward an observation tool 7, namely an imaging device, in the X direction utilizing the carrying device 9.

The received discrete support 2′ with the flow cell 2 is assignable to its original position on the work area 4 in accordance with the X position of carrying plate 11. This detection of the X position of the carrying plate 11 and of the movement path of the gripping mechanism 8 to grasp the object (original Y position of the object) is performed via suitable sensors (not shown) for detecting linear movements, as are known to those skilled in the art from the relevant related art. The processing of the information from these sensors, the control of the drives for the movement of the carrying plate 11 in the X direction and the gripping mechanism 8 in the Y direction, and the assignment of this information to an original X/Y position of the object is preferably performed using a suitably programmed controller implemented in a digital computer (not shown), which is also a coupled part of the system.

Since in the sequencing of unknown nucleic acids, all samples contained within the flow cells will be to some degree variable, the identification of all flow cell supports 2 of the entire platform 3 is desirable and advantageous. It may also be important to track individual sequences of a series of flow cells via software applications. The defined position and orientation of the flow cells on the reaction platform allow identification of each set of sequencing samples, and thus tracking of the samples for purposes of later cross-checking and assembly.

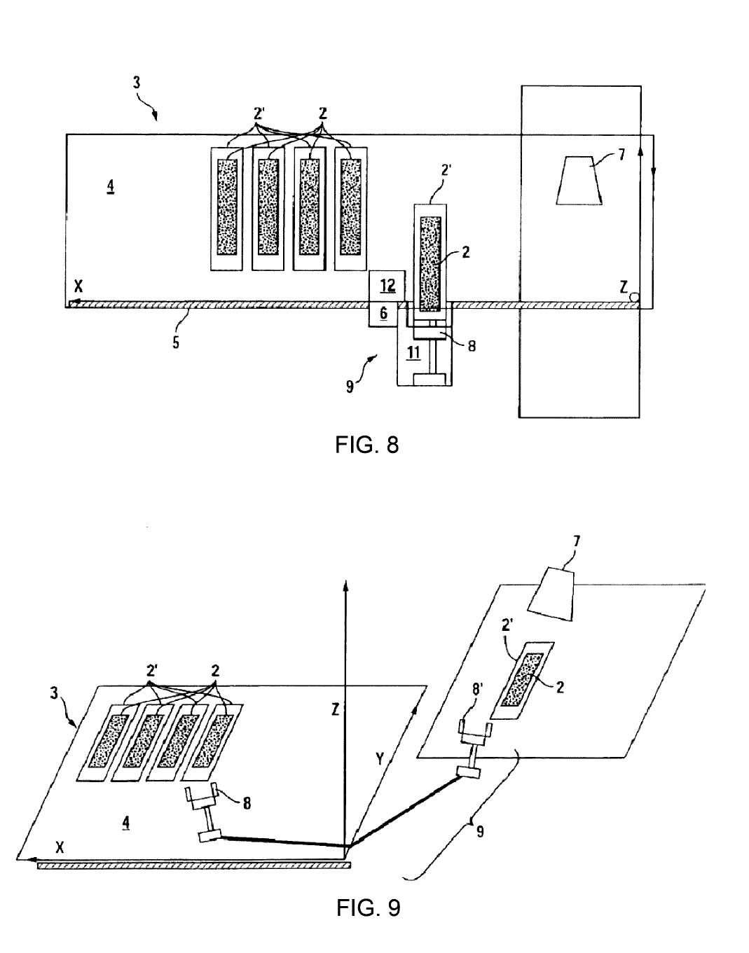

In specific aspects of these embodiments, the flow cell 2 and the support 2′ are formed as a single, integrated construct. In a specific embodiment illustrated in FIG. 8, the system 3 further provides a characterization tool 12, such as a barcode reader. This characterization tool can read one or more identifying elements of a support 2′ and determine the identity of the samples in the corresponding flow cell. This identification is preferably performed while the support 2′ is pulled onto the carrying plate 11 of the carrying device 9.

FIG. 9 illustrates the support 2′ comprising the flow cell 2 as transferred in the Z direction to an imaging system in a plane different than that of the reaction platform 3. The gripping mechanism comprising element 8 in one position and 8′ in another position is implemented here as a telescopic arm; as an alternative to this, it may also be implemented as an articulated arm. The carrying device 9 is rotatable around an angle, which is preferably +180° and/or −180°, in relation to a Z axis perpendicular to the horizontal work field 4. A further alternative embodiment of the gripping mechanism (not shown) includes a rail running in the Y direction having a caterpillar tread, which may be raised and/or lowered in order to grasp and/or deposit the carrier, for example. Using this carrying device 9, the support 2′ comprising the flow cell 2 may be transferred in the X direction and then deposited using the gripping mechanism 8 at a position within the viewing area of the observation tool 7, which is different from the original position of the object on the work area 4, which is significantly, the area where the chemistry is performed prior to observation. At the same time, as the gripping mechanism 8 is moved out, the identity of the samples and/or the objects is preferably detected once again and the new X/Y position of the flow cell 2 and the support 2′ is stored in the coupled computer component of the system.

From the previous description, it may be seen that the support 2′ may not only be grasped, transferred in a plane, and deposited again using the gripping mechanism 8′, the support 2′ may also be transferred from one plane to a plane positioned above or below it in the Z direction and deposited there for further analysis using an illumination, detection and analysis component of the system of the invention. As these transfer tasks are executed, it is advantageous, but not absolutely necessary, for each of the objects to be identified or otherwise characterized using the characterization tool 12 (FIG. 8).

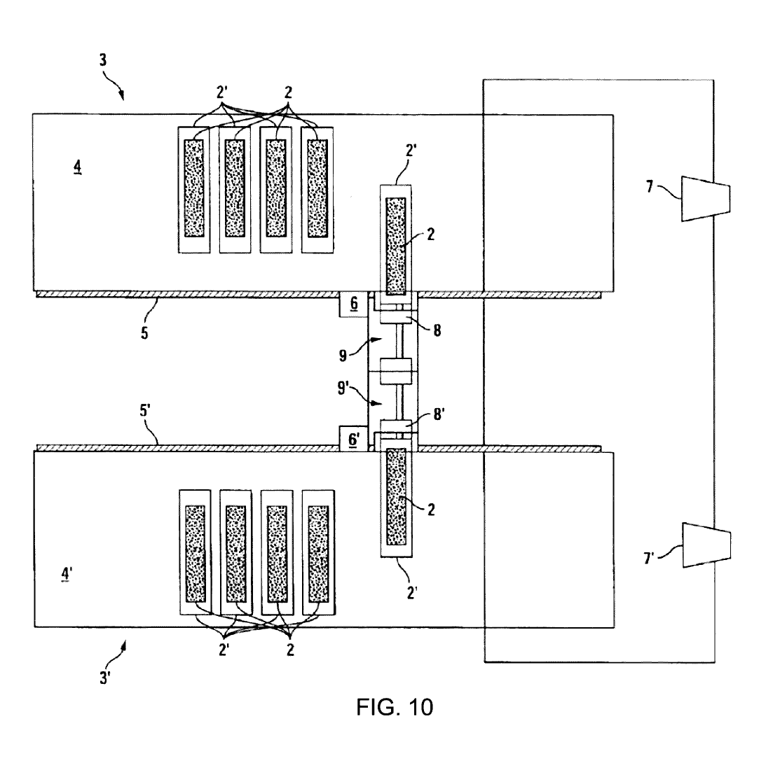

More than two work platforms may be combined into a higher-order system, as illustrated in FIG. 10. The work fields 4, 4′ may be positioned parallel to one another, end-to-end in series or one over another and rotated by an arbitrary angle in a horizontal plane (not illustrated).

An aspect of the invention is timely and efficient support for the automated sequencing of reaction components. This process may involve a plurality of sequencing reactions system components that are optimized for the biochemical interrogation of nucleic acids of unknown sequence. A variety of biochemical sequencing reactions can be used with the systems of the invention, including, but not limited to, hybridization-based methods, such as disclosed in U.S. Pat. Nos. 6,864,052; 6,309,824; and 6,401,267 and U.S. patent publication 2005/0191656; sequencing by synthesis methods, such as disclosed in U.S. Pat. Nos. 6,210,891; 6,828,100; 6,833,246; 6,911,345; articles Ronaghi et al (1998), Science, 281:363-365; and Li et al, Proc. Natl. Acad. Sci., 100:414-419 (2003); and ligation-based methods, as disclosed e.g., in International Patent applications WO1999019341, WO2005082098, WO2006073504 and article Shendure et al (2005), Science, 309:1728-1739.

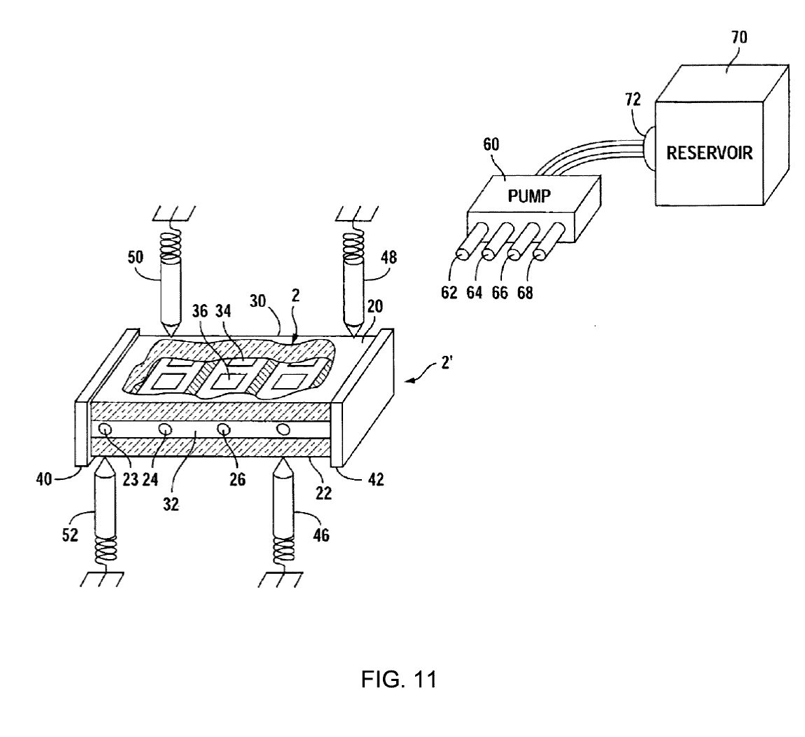

In particular embodiments, the sequencing reaction component of the system comprises one or more flow cells 2 (i.e., reaction chambers) (FIG. 11) in which the actual biochemical sequencing reaction takes place. In preferred embodiments of the invention, the flow cells 2 of the sequencing reaction component of the system comprise chambers in a support structure 2′ for example constructed of optical microscope slides 20, 22 spaced by spacers 23, 24, 26, 28 into flow cell chambers 2, each with an inlet 30, an outlet 32, and a surface with exemplary regions 34, 36 that have been manufactured or otherwise treated to allow nucleic acids to be attached thereto when injected in a liquid transport through the inlet 30. The flow cell optionally includes nucleic acids or primers attached to surface regions 34, 36 of the flow cell, either as a random array or in a predetermined array of microsites so that the identity of each nucleic acid can be monitored throughout the reaction process. Nucleic acids or primers can be attached to the surface such that at least a portion of the nucleic acids or primers are individually optically resolvable when viewed through the walls of the support structure 2′.

In one preferred embodiment, the flow cells 2 comprise a substantially sealed chamber having a solid support or at least a backing on which nucleic acids of unknown sequence are immobilized. The flow cells 2 are preferably associated with a support retaining member (table or cassette) for placement of the solid support or backing in the sequencing reaction component of the system. The flow cells 2 may, for example, be arranged side-by-side, or one in front of the other on the sequencing reaction system component. Where the solid support 2′ comprises is a microscope slide 22, the support retaining member will typically be of such dimensions that it may be used with slides of conventional size, (i.e., slides which typically are about 25.4 mm by 76.2 mm). Where the support is a membrane, the dimensions of the retaining member will similarly be of such dimensions that it may be used with membranes of conventional size (typically 80 mm by 120 mm), although membranes are rather more variable in size than slides.

The structural aspects of the flow cell are typically held together by an adhesive (associated with spacer elements 23, 24, 26, 28) or by a clamping means 40, 42. In certain aspects of the embodiments of the invention, the clamping means 40, 42 is capable of clamping together the portions of a plurality of flow cells. Typically, from one to around twelve or sixteen flow cells may be clamped simultaneously by a single clamping means. The flow cells can be arranged in the clamping means in a substantially horizontal or substantially vertical manner, although any position intermediate between these two positions is possible.

As an alternative or in addition to clamping, the flow cell may be provided with a biasing structure that joins the components of the flow cell. The biasing structure may comprise one or more sprung biasing members 46, 48, 50, 52. In a particular embodiment, the support is attached to a clamp by spring-loaded mounting pins, such that formation of the flow cell places the springs of the spring-loaded mounting pins under compression, which springs therefore connect the components of the flow cell.

In other specific aspects of the embodiments of the invention, the force applied to the flow cell structure by the clamping means and/or the biasing means helps to ensure a fluid-tight seal between the support and the support retaining member.

In certain aspects, it is generally preferred that the flow cell additionally comprises sealing means to assist in the formation of a substantially sealed chamber. The sealing means may be an integral part of the support retaining member, or may be provided as a separate component of the flow cell. The sealing means typically comprises a gasket, which may be made of silicon rubber or other suitable material. In one embodiment the sealing means comprises an O-ring gasket, the shape of which is generally that of a frame-like surround seated in a groove in one portion of the support retaining member. In an alternative embodiment the sealing means comprises a flattened frame-like surround gasket (about 100 to 150 μm thick). In other specific aspects, a gasket or other spacer material can be attached with an adhesive.

Either type of gasket may be discarded after a single use (if, for example, contaminated with a radioactive probe) or may be re-used if desired. The flattened gasket embodiment is particularly suitable as a disposable gasket, to be discarded after a single use. It will be apparent that the thickness of the gasket (which can be readily altered by exchanging gaskets) may, in part, determine the volume of the substantially sealed chamber.

In another aspect of the invention using small volumes in the sequencing reactions, the flow cell components are directly connected via the use of an adhesive. The adhesive is preferably introduced to a surface that provides optimal adhesion between the various flow cell components, e.g., a slide comprising an array and a coverslip.

The fluid inlet 30 allows the introduction into the substantially sealed chamber of fluids needed to process the sample on the support. Typically such fluids will be buffers, solvents (e.g. ethanol/methanol, xylene), reagents (e.g., primer- or probe-containing solutions) and the like. The fluid outlet allows for the processing fluids to be removed from the sample (e.g., for washing, or to allow the addition of a further reagent). Preferably, when the supports are being processed, their orientation is such that the fluid inlet is in the bottom portion of the substantially sealed chamber, and the fluid outlet is in the top portion of the substantially sealed chamber.

Typically, where the nucleic acid sample is supported on a slide 22, the substantially sealed chamber will have a volume of between 50 μl and 300 μl, preferably between 100-150 μl. This small volume allows for economical use of reagents and (where temperature regulation is involved) a rapid thermal response time. Where the sample is supported on a membrane, the chamber will generally be larger (up to 2-3 mls).

In particular aspects, the flow cell 2 is adapted so as to be suitable for use in performing amplification (e.g., rolling circle amplification or polymerase chain reaction amplification) on samples attached to a support. In such an embodiment, the flow cell must have an opening to allow the addition of further reagents. This opening must be designed so that it is transitory and the flow of any new liquids is very tightly controlled to prevent any leakage from the flow cell and to prevent contamination of the flow cell upon addition of any new reagents.

In a particular aspects of certain embodiments, for example those envisaged for use with PCR or other reactions in which tightly controlled temperature regulation is required, the flow cell is equipped with temperature control means to allow for rapid heating and cooling of the sample and PCR mix (i.e. thermal cycling). Typically the flow cell will be provided with an electrical heating element or a Peltier device. The flow cell may also be adapted (e.g., by provision of cooling means) to provide for improved air cooling. Temperature control in the range 3°-105° C. is sufficient for most applications.

A number of arrangements for appropriate fluid delivery means can be envisaged. In a preferred embodiment a number of reservoirs of processing fluids, (e.g., buffers, stains, etc.) are provided, each reservoir being attached to a pumping mechanism. Preferred pumping mechanisms include, but are not limited to syringe pumps 60, such as those manufactured by Hook and Tucker, (Croydon, Surrey, UK), or Kloen having a stroke volume of between 1 and 10 ml. One such pump 60 may be provided for each processing fluid reservoir, or a single pump may be provided to pump fluid from each a plurality of reservoirs, by means of a multi-port valve configuration to a plurality of syringe needles 62, 64, 66, 68 alignable with the inlets 30.

Each syringe pump 60 can in turn be attached such as by a universal connector to a central manifold 70 (such as a universal connector). Preferably the central manifold 70 feeds into a selective multi-outlet valve 72 such that, if desired, where a plurality of samples are being processed simultaneously, each sample may be treated with a different processing fluid or combination of processing fluids. A suitable selective multi-outlet valve is a rotary valve, such as the 10 outlet rotary valve supplied by Omnifit (Cambridge, UK). Thus each outlet from the multi-outlet valve 72 may be connected to a separate flow cell. One or more filters may be incorporated if desired. Typically a filter will be positioned between each reservoir and its associated syringe pump.

Each syringe pump 60 may be actuated individually by the computer control means, or two or more pumps may be actuated simultaneously to provide a mixture of two or more processing fluids. Controlling the rate of operation of each pump 60 will thus control the composition of the resulting mixture of processing fluids.

In an alternative embodiment, the fluid delivery means comprises two or more piston/HPLC-type pumps, each pump being supplied, via a multi-inlet valve, by a plurality of processing fluid reservoirs. Suitable pumps are available, for example, from Anachem (Luton, Beds, UK). The multi-inlet valve will be a rotary valve. Each pump will feed into a rotary mixer, of the type well known to those skilled in the art, thus allowing variable composition mixtures of processing fluids to be produced, if desired.

In certain aspects, the processing fluid or mixture of processing fluids is then passed through an in-line filter and then passes through a selective multi-valve outlet (such as a rotary valve) before being fed into the flow cells.

As an alternative to the generally “parallel” supply of processing fluids defined above, the processing fluids may be supplied in “series” such that, for example, fluid is passed from one substantially sealed chamber to another. This embodiment has the advantage that the amount of reagent required is minimized.

In aspects of the invention comprising one or more valves, typically the valve will be a three-way valve with two inlets, and one outlet leading to the substantially sealed flow cell. One of the valve inlets is fed, indirectly, by the reservoirs of processing fluid. The second inlet is fed by a local reservoir which, typically, will be a syringe, pipette or micro-pipette (generally 100-5000 μl volume). This local reservoir may be controlled by the computer control means or may be manually controlled. The local reservoir will typically be used where a reagent is scarce or expensive. The provision of such a local reservoir minimizes the amount of reagent required, simplifies cleaning, and provides extra flexibility in that each flow cell may be processed individually, if required.

In a specific aspect of certain embodiments of the invention, the “flow” for use in the flow cell reaction is achieved by gravity force, e.g., placement of the flow cell at an angle or by the use of an absorbent material applied on the outlet 32 of the flow cell. In other aspects of the embodiments, the flow is produced using either mechanical or electrical means, e.g., the introduction of a vacuum apparatus to the outlet edge of the flow cell. The flow cell in such embodiments may be substantially sealed, or may have both an inlet and an outlet available for transfer of fluids through the flow cell.

In another specific aspect of the embodiments of the invention, fluid enters the flow cell at the bottom, travel upwards and exits from the flow cell via the fluid outlet at the top. In a preferred aspect, however, fluid enters the flow cell from the top and is carried through the reaction via gravity, exiting the flow cell via a fluid outlet at the bottom. The fluid outlet can empty into a common collecting duct, which duct drains into a collecting vessel. The vessel is desirably removable from the apparatus to allow for periodic emptying and/or cleaning.

According to the invention, to accommodate various incompatible reaction speeds and volumes of material to be processed, the sequencing reaction component is substantially modular such that, should large numbers of flow cells and/or supported samples require processing, additional elements can be readily added to the existing equipment. In such an embodiment, the observation component as well as the sequencing reaction component of the system are preferably capable of accepting a modular array of flow cells, whether the samples are supported on slides or membranes.

The reversible integration of the sequencing reaction component to the system may include a connection to a computer control means, which can coordinate the different activities of the functional elements of the system. The computer control means can optionally control two or more of the following parameters: the selection of which pump or pumps to actuate; the absolute volume and the rate of flow of processing fluid passing through the actuated pump(s); the selection of which flow cell to feed with processing fluid; the temperature of the supported samples within the apparatus; movement of the flow cell from the sequencing reaction apparatus to the imaging component of the system; and the timing of the various events.

The invention further relates to manufacture of and use of the flow cell and/or the apparatus of the invention in processing a sample on a support, such that the invention provides: a method of processing a sample on a support using a flow cell and/or the automated sequencing reaction apparatus defined above; a method of making a flow cell; and a method of making a loosely-coupled, reversibly integrated system comprising a sequencing reaction component in accordance with the present invention.

The present invention provides a detection component for the identification of the results of the sequencing reaction component of the systems of the invention. The detection system for the signal may depend upon the labeling moiety used, which can be defined by the chemistry available. Any detection method may be used that is suitable for the type of label employed can be used in the detection component of the systems of the invention. Thus, exemplary detection methods include radioactive detection, optical absorbance detection, e.g., UV-visible absorbance detection, optical emission detection, e.g., fluorescence or chemiluminescence. Optical setups include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy.

Labeled nucleic acid molecules can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Guidance can be found in the literature for applying such techniques for analyzing and detecting nanoscale structures on surfaces, as evidenced by the following references: Reimer et al, editors, Scanning Electron Microscopy: Physics of Image Formation and Microanalysis, 2nd Edition (Springer, 1998); Nie et al, Anal. Chem., 78: 1528-1534 (2006); Hecht et al, Journal Chemical Physics, 112: 7761-7774 (2000); Zhu et al, editors, Near-Field Optics: Principles and Applications (World Scientific Publishing, Singapore, 1999); Drmanac, International patent publication WO 2004/076683; Lehr et al, Anal. Chem., 75: 2414-2420 (2003); Neuschafer et al, Biosensors & Bioelectronics, 18: 489-497 (2003); Neuschafer et al, U.S. Pat. No. 6,289,144; and the like.

One specific imaging technique for use in the present invention is total internal reflection fluorescence (TIRF) microscopy, which can be used to visualize single fluorophores (Cy-3 or Cy-5 labeled dNTPs). TIRF microscopy uses totally internally reflected excitation light, and detection is generally carried out using evanescent wave illumination and TIRF microscopy. An evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. In other words, the optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths. Examples of this technique are disclosed by Neuschafer et al, U.S. Pat. No. 6,289,144; Lehr et al (cited above); and Drmanac, International patent publication WO 2004/076683.

EPI-fluorescence illumination can also be employed in the detection component of the invention. EPI-fluorescence microscopy is a technique which involves staining with a special type of histological stain called a fluorochrome which is taken up during hybridization of fluorescently labeled complementary DNA sequences.

[Both TIRF and EPI illumination allow for almost any light source to be used. The light source can be rastered, spread beam, coherent, incoherent, and originate from a single or multi-spectrum source. In one specific aspect of the embodiments, imaging may be accomplished with a 100× objective using TIRF or EPI illumination and a 1.3 mega pixel Hamamatsu orca-er-ag on a Zeiss axiovert 200, or like system component.

Fluorescence resonance energy transfer (FRET) can also be used as a detection scheme. FRET in the context of sequencing is described generally in Braslavasky, et al., Proc. Nat'l Acad. Sci., 100: 3960-3964 (2003), incorporated by reference herein. Essentially, in one embodiment, a donor fluorophore is attached to the primer, polymerase, or template. Nucleotides added for incorporation into the primer comprise an acceptor fluorophore that is activated by the donor when the two are in proximity.

A suitable illumination and detection system for fluorescence-based signal is a Zeiss Axiovert 200 equipped with a TIRF slider coupled to a 80 milliwatt 532 nm solid state laser. The slider illuminates the substrate through the objective at the correct TIRF illumination angle. TIRF can also be accomplished without the use of the objective by illuminating the substrate though a prism optically coupled to the substrate. Planar wave guides can also be used to implement TIRF on the substrate.

One embodiment for the imaging system contains a 20× lens with a 1.25 mm field of view, with detection being accomplished with a 10 megapixel camera. Such a system images approx 1.5 million nucleic acid molecules attached to the patterned array at 1 micron pitch. Under this configuration there are approximately 6.4 pixels per nucleic acid molecule. The number of pixels per nucleic acid molecule can be adjusted by increasing or decreasing the field of view of the objective. For example a 1 mm field of view would yield a value of 10 pixels per nucleic acid molecule and a 2 mm field of view would yield a value of 2.5 pixels per nucleic acid molecule. The field of view may be adjusted relative to the magnification and NA of the objective to yield the lowest pixel count nucleic acid molecule that is still capable of being resolved by the optics, and image analysis software. Imaging speed may be improved by decreasing the objective magnification power, using grid patterned arrays and increasing the number of pixels of data collected in each image.

For optical signals, a combination of an optical fiber or charged couple device (CCD) can be used in the detection of the sequencing reaction. Thus, in particular embodiments, the hybridization patterns on the array formed from the sequencing reactions are scanned using a CCD camera (e.g., Model TE/CCD512SF, Princeton Instruments, Trenton, N.J.) with suitable optics (Ploem, in Fluorescent and Luminescent Probes for Biological Activity Mason, T. G. Ed., Academic Press, Landon, pp. 1-11 (1993), such as described in Yershov et al., Proc. Natl. Aca. Sci. 93:4913 (1996), which allows simultaneous scanning of a very high number of labeled target nucleic acids.

In specific embodiments, the efficiency of the sequencing system can be enhanced through the use of a multi-imaging system component. For example, up to four or more cameras may be used in the imaging component of the system, preferably in the 10-16 megapixel range. Multiple band pass filters and dichroic mirrors may also be used to collect pixel data across up to four or more emission spectra. To compensate for the lower light collecting power of the decreased magnification objective, the power of the excitation light source can be increased. Throughput can be increased by using one or more flow cells with each camera, so that the imaging system is not idle while the samples are being hybridized/reacted. Because the probing of arrays can be non-sequential, more than one imaging system can be used to collect data from a set of arrays, further decreasing assay time.

One illumination schema is to share a common set of monochromatic illumination sources (about four lasers for 6-8 colors) amongst imagers. Each imager collects data at a different wavelength at any given time and the light sources would be switched to the imagers via an optical switching system. In such an embodiment, the illumination source preferably produces at least six, but more preferably eight different wavelengths. Such sources include gas lasers, multiple diode pumped solid state lasers combined through a fiber coupler, filtered Xenon Arc lamps, tunable lasers, or the more novel Spectralum Light Engine, soon to be offered by Tidal Photonics. The Spectralum Light Engine uses prism to spectrally separate light. The spectrum is projected onto a Texas Instruments Digital Light Processor, which can selectively reflect any portion of the spectrum into a fiber or optical connector. This system is capable of monitoring and calibrating the power output across individual wavelengths to keep them constant so as to automatically compensate for intensity differences as bulbs age or between bulb changes.

During the imaging process, the substrate must remain in focus. Some key factors in maintaining focus are the flatness of the substrate, orthogonality of the substrate to the focus plane, and mechanical forces on the substrate that may deform it. Substrate flatness can be well controlled, as glass plates which have better than ¼ wave flatness are readily obtained. Uneven mechanical forces on the substrate can be minimized through proper design of the hybridization chamber. Orthogonality to the focus plane can be achieved by a well adjusted, high-precision stage. After each image is acquired, it will be analyzed using a fast algorithm to determine if the image is in focus. If the image is out of focus, the auto focus system will store the position information of the out-of-focus image so that section of that array can be re-imaged during the next imaging cycle. By mapping the position at various locations on the substrate, the time required for substrate image acquisition can be reduced.

Measured signals can be analyzed manually or, preferably, by appropriate computer methods to tabulate results. The substrates and reaction conditions can include appropriate controls for verifying the integrity of hybridization and extension conditions, and for providing standard curves for quantification, if desired. For example, a control nucleic acid can be added to the sample.

In a large scale sequencing operation, each imager preferably acquires ˜200,000 images per day, based on a 300 millisecond exposure time to a 16 mega pixel CCD. Thus, an instrument design for the illumination and detection component of the system of the invention may comprise four imager modules each serving four sets of quad flow cells (16 flow cells total). Each imager can include a CCD detector with 10 million pixels and be used with an exposure time of roughly 300 milliseconds. Unintentionally photo bleaching by the light source while other fluorophores are being imaged can be reduced by keeping the illumination power low and exposure times to a minimum.

By using intensified CCDs (ICCDs), data is collected of roughly the same quality with illumination intensities and exposure times that are orders of magnitude lower than standard CCDs. ICCDs are generally available in the 1-1.4 megapixel range. Because they require much shorter exposure times, a one megapixel ICCD can acquire ten or more images in the time a standard CCD acquires a single image. Used in conjunction with fast filter wheels, and a high speed flow cell stage, a one mega pixel ICCD can collect the same amount of data as a 10 megapixel standard CCD.

In a specific embodiment, an electron multiplying CCD (EMCCD) is used to image the nucleic acids. The EMCCD is a quantitative digital camera technology that is capable of detecting single photon events whilst maintaining high quantum efficiency, achievable by way of a unique electron multiplying structure built into the sensor. Unlike a conventional CCD, an EMCCD is not limited by the readout noise of the output amplifier, even when operated at high readout speeds. This is achieved by adding a solid state Electron Multiplying (EM) register to the end of the normal serial register; this register allows weak signals to be multiplied before any readout noise is added by the output amplifier, hence rendering the read noise negligible. The EM register has several hundred stages that use higher than normal clock voltages. As charge is transferred through each stage the phenomenon of Impact Ionization is utilized to produce secondary electrons, and hence EM gain. When this is done over several hundred stages, the resultant gain can be (software) controlled from unity to hundreds or even thousands of times.

The EMCCD system can be used in conjunction with a TIFRM technique to image multiple fluorophore labels, through integration of a multi-line laser system, preferably a solid-state laser solution with Acousto-Optical Tunable Filter (AOTF) modulation. This technique can be readily adapted for FRET analysis, preferably through integration of a suitable beam splitting device on the emission side.

A factor to be considered in high-resolution and high-speed imaging and readout in connection with sequencing chemistry is the consequence of vibration caused by moving parts, vibrations, which if not controlled or isolated, can disrupt image capture and result in poor image resolution. To minimize the effects of vibrations from moving parts, particularly the carrying tool 9 with the motorized gripping mechanism 8, 8′, the characterization tool 7 comprising the optical components and the reaction platform 3 are specifically loosely coupled physically. In particularly, they are physically isolated from one another by shock isolators or the like, even though they are juxtaposed in operation. This is facilitated by and may require that there be a control and sensing mechanism as part of the carrying tool 9 as well as a position registration mechanism as part of the characterization tool 7. Various such mechanisms are within the teachings of related arts. For example the robotics, wherein electronic eyes are employed, alignment marks and the like that can be sensed are used to assure transfer is accurate without inducing undue vibration into the sensitive field of view of the characterization tool so as to permit continuous or nearly continuous operation. The goal is to collect and process massive amounts of data accurately and with efficiency, while interfacing two or more technologies, involving batch-like processes with mechanical, electronic, optical and biochemical aspects, that have not heretofore been integrated into an efficient continuously operating analytic method.

In summary the invention may be characterized as an integrated, automated nucleic acid sequencing system having a nucleic acid extraction module, wherein a nucleic acid is extracted from a sample that comprises the nucleic acid; a library preparation module, wherein a library of barcoded nucleic acid constructs is prepared from the extracted nucleic acid; a nucleic acid sequencing module comprising a flow cell loader, at least one flow cell, an imager, and at least one liquid handler that performs sequencing reactions, wherein said at least one flow cell comprises a substrate for attachment of the barcoded nucleic acid constructs in an array, the flow cell loader is configured to load the barcoded nucleic acid constructs into the flow cell, the liquid handler is configured to perform nucleic acid sequencing reactions on the barcoded nucleic acid constructs in the array, and the imager is configured to produce images of the barcoded nucleic acid constructs in the array after sequencing; a data analysis module, wherein the images are analyzed to produce reads, the reads are assembled to produce assembled sequence, and variants are identified in the assembled sequence; and a workflow management system comprising a user interface for managing operation of the nucleic acid extraction module, the library preparation module, the nucleic acid sequencing module and the data analysis module.

In specific embodiments, the system's nucleic acid sequencing module includes a plurality of liquid handlers, wherein the liquid handlers operate independently of each other optionally with at least one liquid handler that performs a first type of sequencing reaction and at least one liquid handler that performs a second type of sequencing reaction that differs from the second sequencing reaction. The first type of sequencing reaction may be cPAL sequencing and the second type of sequencing reaction may be sequencing by synthesis. The nucleic acid sequencing module may comprise a plurality of flow cells.

In the system, the liquid handler and the imager are loosely coupled and comprise a carrying device configured for transferring said at least one flow cell from the liquid handler to the imager. The nucleic acid sequencing module and the imager are configured to operate independently at different rates. The nucleic acid sequencing module may include shock isolators that are constructed and arranged so as to sufficiently isolate the imager from vibrations so that the vibrations do not disrupt image capture.

A method for nucleic acid sequencing system includes providing a nucleic acid sequencing system having the foregoing features; extracting a nucleic acid from a sample comprising the nucleic acid using the nucleic acid extraction module; preparing a library of barcoded nucleic acid constructs from the extracted nucleic acid using the library preparation module; loading the library of nucleic acid constructs into said at least one flow cell comprising a substrate for attachment of the constructs in an array using the flow cell loader; performing nucleic acid sequencing reactions on the nucleic acid constructs in said at least one flow cell; producing images of the nucleic acid constructs in the array after sequencing using the imager; performing data analysis using the data analysis module, wherein a basecalling element operating a data processing component produces reads from analysis of the images, sequence assembly element assembles the reads to produce an assembled sequence, and variant identification element identifies variants in the assembled sequence; and managing the workflow from extracting the nucleic acid to data analysis using the workflow management system.

A method of nucleic acid sequencing that has fully automated workflow may include the steps of extracting a nucleic acid from a sample comprising the nucleic acid; preparing a library of barcoded nucleic acid constructs from the extracted nucleic acid; loading the library of nucleic acid constructs into at least one flow cell comprising a substrate for attachment of the constructs in an array; performing nucleic acid sequencing reactions on the nucleic acid constructs in said at least one flow cell; producing images of the nucleic acid constructs after sequencing; performing data analysis comprising producing reads from analysis of the images, assembling the reads to produce an assembled sequence, and identifying variants in the assembled sequence; and managing operation of the workflow using a workflow management system comprising a user interface.

The foregoing method may be further characterized by extracting nucleic acids from a plurality of samples; preparing separate libraries of nucleic acid constructs from each of said plurality of samples; pooling said separate libraries; and loading the pooled libraries into the flow cell. Additionally it may be characterized by loading the pooled libraries into a plurality of flow cells; amplifying the library of nucleic acid constructs to produce DNA nanoballs before loading into said at least one flow cell; or amplifying the nucleic acid constructs after loading into said at least one flow cell.

The foregoing method may be further characterized by the step of performing nucleic acid sequencing reactions on the nucleic acid constructs using one or more liquid handlers, and the step of producing images of the nucleic acid constructs after sequencing is performed using an imager, the method further comprising transferring said at least one flow cell from the liquid handler to the imager using a carrying device; and characterized by the step of performing nucleic acid sequencing reactions on the nucleic acid constructs using a plurality of liquid handlers, the method comprising serially transferring each of the flow cells to an imager for producing images of the nucleic acid constructs after sequencing. Still further, the method may be characterized by the step of performing nucleic acid sequencing reactions in the flow cells and the by step of producing images of the nucleic acid constructs after sequencing operate at different rates.

The method may be characterized in that the step of performing nucleic acid sequencing reactions on the nucleic acid constructs is performed using two or more liquid handlers that operate independently of each other.

In as specific embodiment, an integrated, automated nucleic acid sequencing system includes a nucleic acid extraction module, the nucleic acid extraction module configured to extract a nucleic acid from a sample that comprises the nucleic acid; a library preparation module, configured to prepare a library of barcoded nucleic acid constructs from the extracted nucleic acid; a nucleic acid sequencing module reversibly integrated with the library preparation module and the library preparation module and comprising components reversibl integrated with one another, the nucleic acid sequencing module components comprising a flow cell loader, at least one flow cell removably attachable to the flow cell loader, an imager configured to view the at least one flow cell, a robot configured to transport the at least one flow cell between the imager and a liquid carrying tool, and at least one liquid carrying tool coupled with the robot and physically loosely coupled with the imager by physical and vibration isolation, the liquid carrying tool including a motion control and position sensing mechanism, the carrying tool being configured to handle the at least one flow cell, the at least one flow cell being the mechanism in which sequencing reactions are performed, wherein the flow cell comprises a substrate configured for attachment of the barcoded nucleic acid constructs in an array, the loader configured to load the barcoded nucleic acid constructs into the flow cell, the liquid carrying tool configured to perform nucleic acid sequencing reactions on the barcoded nucleic acid constructs in the array of the flow cell, and the imager configured to produce images of the barcoded nucleic acid constructs in the array after sequencing; a data analysis module reversibly integrated with the nucleic acid sequencing module including a position registration mechanism configured to register positioning of the array of the flow cell and comprising data processing elements configured to perform basecalling from data extracted from the images, wherein the images are analyzed to produce reads, sequence assembly, wherein the reads are assembled, and variant identification, and wherein variants are identified in the assembled sequence; and a workflow management system reversibly integrated with the nucleic acid extraction module, the library preparation module, the nucleic acid sequencing module, and the data analysis module and comprising input/output components providing a user interface for managing the operation of the nucleic acid extraction module, the library preparation module, the nucleic acid sequencing module and the data analysis module.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured only by claims of any corresponding utility application and their equivalents. The abstract and the title are not to be construed as limiting the scope of the present invention, as their purpose is to enable the appropriate authorities, as well as the general public, to quickly determine the general nature of the invention. In the claims of any corresponding utility application, unless the term “means” is used, none of the features or elements recited therein should be construed as means-plus-function limitations pursuant to 35 U.S.C. §112, ¶6.

What is claimed is:

1. An integrated, automated nucleic acid sequencing system comprising:
a nucleic acid extraction module, wherein a nucleic acid is extracted from a sample that comprises the nucleic acid;
a library preparation module, wherein a library of barcoded nucleic acid constructs is prepared from the extracted nucleic acid;
a nucleic acid sequencing module comprising a flow cell loader, at least one flow cell, an imager, and at least one liquid handler that performs sequencing reactions, wherein
said at least one flow cell comprises a substrate for attachment of the barcoded nucleic acid constructs in an array,
the flow cell loader is configured to load the barcoded nucleic acid constructs into the flow cell,
the liquid handler is configured to perform nucleic acid sequencing reactions on the barcoded nucleic acid constructs in the array, and
the imager is configured to produce images of the barcoded nucleic acid constructs in the array after sequencing;
a data analysis module, wherein
the images are analyzed to produce reads, the reads are assembled to produce assembled sequence, and variants are identified in the assembled sequence; and
a workflow management system comprising a user interface for managing operation of the nucleic acid extraction module, the library preparation module, the nucleic acid sequencing module and the data analysis module.

2. The system of claim 1 wherein the nucleic acid sequencing module comprises a plurality of liquid handlers.

3. The system of claim 2 wherein the liquid handlers operate independently of each other.

4. The system of claim 3 comprising at least one liquid handler that performs a first type of sequencing reaction and at least one liquid handler that performs a second type of sequencing reaction that differs from the second sequencing reaction.

5. The system of claim 4 wherein the first type of sequencing reaction is cPAL sequencing and the second type of sequencing reaction is sequencing by synthesis.

6. The system of claim 1 wherein the nucleic acid sequencing module comprises a plurality of flow cells.

7. The system of claim 1 wherein the liquid handler and the imager are loosely coupled, the system comprising a carrying device configured for transferring said at least one flow cell from the liquid handler to the imager.

8. The system of claim 1 wherein the nucleic acid sequencing module and the imager are configured to operate independently at different rates.

9. The system of claim 1 wherein the nucleic acid sequencing module comprises shock isolators that are constructed and arranged so as to sufficiently isolate the imager from vibrations so that the vibrations do not disrupt image capture.

10. A method for nucleic acid sequencing system according to claim 1 comprising:
providing a nucleic acid sequencing system of claim 1;
extracting a nucleic acid from a sample comprising the nucleic acid using the nucleic acid extraction module;
preparing a library of barcoded nucleic acid constructs from the extracted nucleic acid using the library preparation module;
loading the library of nucleic acid constructs into said at least one flow cell comprising a substrate for attachment of the constructs in an array using the flow cell loader;
performing nucleic acid sequencing reactions on the nucleic acid constructs in said at least one flow cell;
producing images of the nucleic acid constructs in the array after sequencing using the imager;
performing data analysis using the data analysis module, wherein a basecalling element operating a data processing component produces reads from analysis of the images, a sequence assembly element assembles the reads to produce an assembled sequence, and a variant identification element identifies variants in the assembled sequence; and
managing the workflow from extracting the nucleic acid to data analysis using the workflow management system.

11. A method of nucleic acid sequencing comprising a fully automated workflow that comprises the steps of:
extracting a nucleic acid from a sample comprising the nucleic acid;
preparing a library of barcoded nucleic acid constructs from the extracted nucleic acid;
loading the library of nucleic acid constructs into at least one flow cell comprising a substrate for attachment of the constructs in an array;
performing nucleic acid sequencing reactions on the nucleic acid constructs in said at least one flow cell;
producing images of the nucleic acid constructs after sequencing;
performing data analysis comprising producing reads from analysis of the images, assembling the reads to produce an assembled sequence, and identifying variants in the assembled sequence; and
managing operation of the workflow using a workflow management system comprising a user interface.

12. The method of claim 11 comprising:
extracting nucleic acids from a plurality of samples;
preparing separate libraries of nucleic acid constructs from each of said plurality of samples;
pooling said separate libraries; and
loading the pooled libraries into the flow cell.

13. The method of claim 12 comprising loading the pooled libraries into a plurality of flow cells.

14. The method of claim 11 comprising amplifying the library of nucleic acid constructs to produce DNA nanoballs before loading into said at least one flow cell.

15. The method of claim 11 comprising amplifying the nucleic acid constructs after loading into said at least one flow cell.

16. The method of claim 11 wherein the step of performing nucleic acid sequencing reactions on the nucleic acid constructs is performed using one or more liquid handlers, and the step of producing images of the nucleic acid constructs after sequencing is performed using an imager, the method further comprising transferring said at least one flow cell from the liquid handler to the imager using a carrying device.

17. The method of claim 16 wherein the step of performing nucleic acid sequencing reactions on the nucleic acid constructs is performed using a plurality of liquid handlers, the method comprising serially transferring each of the flow cells to an imager for producing images of the nucleic acid constructs after sequencing.

18. The method of claim 17 wherein the step of performing nucleic acid sequencing reactions in the flow cells and the step of producing images of the nucleic acid constructs after sequencing operate at different rates.

19. The method of claim 16 wherein the step of performing nucleic acid sequencing reactions on the nucleic acid constructs is performed using two or more liquid handlers that operate independently of each other.

20. An integrated, automated nucleic acid sequencing system comprising:
a nucleic acid extraction module, the nucleic acid extraction module configured to extract a nucleic acid from a sample that comprises the nucleic acid;
a library preparation module, configured to prepare a library of barcoded nucleic acid constructs from the extracted nucleic acid;
a nucleic acid sequencing module reversibly integrated with the library preparation module and the library preparation module and comprising components reversibl integrated with one another, the nucleic acid sequencing module components comprising a flow cell loader, at least one flow cell removably attachable to the flow cell loader, an imager configured to view the at least one flow cell, a robot configured to transport the at least one flow cell between the imager and a liquid carrying tool, and at least one liquid carrying tool coupled with the robot and physically loosely coupled with the imager by physical and vibration isolation, the liquid carrying tool including a motion control and position sensing mechanism, the carrying tool being configured to handle the at least one flow cell, the at least one flow cell being the mechanism in which sequencing reactions are performed, wherein the flow cell comprises a substrate configured for attachment of the barcoded nucleic acid constructs in an array, the loader configured to load the barcoded nucleic acid constructs into the flow cell, the liquid carrying tool configured to perform nucleic acid sequencing reactions on the barcoded nucleic acid constructs in the array of the flow cell, and the imager configured to produce images of the barcoded nucleic acid constructs in the array after sequencing;
a data analysis module reversibly integrated with the nucleic acid sequencing module including a position registration mechanism configured to register positioning of the array of the flow cell and comprising data processing elements configured to perform basecalling from data extracted from the images, wherein the images are analyzed to produce reads, sequence assembly, wherein the reads are assembled, and variant identification, and wherein variants are identified in the assembled sequence; and
a workflow management system reversibly integrated with the nucleic acid extraction module, the library preparation module, the nucleic acid sequencing module, and the data analysis module and comprising input/output components providing a user interface for managing the operation of the nucleic acid extraction module, the library preparation module, the nucleic acid sequencing module and the data analysis module.
